(19) United States
(12) Patent Application Publication
(10) Pub. No.: US 2025/0291015 A1
(43) Pub. Date: Sep. 18, 2025

(54) METHOD FOR ACQUIRING A MAGNETIC RESONANCE IMAGE DATASET

(71) Applicants: Siemens Healthineers AG, Forchheim (DE); The General Hospital Corporation, Boston, MA (US)

(72) Inventors: Daniel Polak, Erlangen (DE); Jeanette Deck, Glattbrugg (CH); Wei Liu, Erlangen (DE); Daniel Nicolas Splitthoff, Uttenreuth (DE); Stephen Farman Cauley, Winchester, MA (US); Lawrence L. Wald, Cambridge, MA (US)

(21) Appl. No.: 19/074,726

(22) Filed: Mar. 10, 2025

(30) Foreign Application Priority Data
Mar. 15, 2024 (EP) 24163711

(51) Int. Cl. G01R 33/561; A61B 5/055; G01R 33/56; G01R 33/565

(52) U.S. Cl. CPC G01R 33/5616; G01R 33/5602; G01R 33/5613; G01R 33/56509; A61B 5/055

(57) ABSTRACT

A method for acquiring a magnetic resonance image dataset of a field-of-view using a gradient-echo imaging protocol includes acquiring additional k-space lines within a central region of k-space at intervals throughout the imaging protocol, wherein the additional k-space lines are used for estimating motion parameters of the field-of-view. The imaging protocol has been amended by inserting additional gradient blips after at least some of the RF excitations, such that at least one additional gradient echo is generated, allowing the acquisition of at least one additional k-space line during one echo time.

The present patent document claims the benefit of European Patent Application No. 24163711, filed Mar. 15, 2024, which is hereby incorporated by reference in its entirety.

### TECHNICAL FIELD

The disclosure relates to a method for acquiring a magnetic resonance image dataset, to a magnetic resonance imaging apparatus, and to a computer program.

### BACKGROUND

Patient motion is one of the most common and costly of artefacts in Magnetic Resonance Imaging (MRI) and may seriously degrade the diagnostic quality of magnetic resonance (MR) exams.

Fast imaging protocols reduce the impact of motion. For example, parallel imaging techniques, as summarized in J. Hamilton, D. Franson, and N. Seiberlich “Recent Advances in Parallel Imaging for MRI,” Prog. Nucl. Magn. Reson. Spectrosc., vol. 101, pp. 71-95, 2017, exploit the properties of modern multi-channel coil arrays to separate aliased pixels in the image domain or to estimate missing k-space data, using knowledge of nearby acquired k-space points, in order to allow scan time reduction by sampling a smaller number of phase encoding lines in k-space.

Some magnetic resonance imaging (MRI) motion correction techniques involve measuring the motion by tracking devices or navigator acquisitions.

By contrast, retrospective motion correction techniques correct for motion artefacts after the data. By including motion parameters into the MR forward model, these techniques account for the patient's motion in the final image reconstruction and therefore reduce motion artefacts. In some techniques, the motion data may be derived from the acquired k-space data itself. For multi-shot acquisitions, the goal in retrospective motion correction techniques is to extract the per shot motion parameters and the motion-free image simultaneously. This may be accomplished by either minimizing an image quality metric, such as image entropy, or by minimizing the data consistency error of a parallel “imaging+motion” forward model, as described in L. Cordero-Grande, E. J. Hughes, J. Hutter, A. N. Price, and J. V Hajnal, “Three-dimensional motion corrected sensitivity encoding reconstruction for multi-shot multi-slice MRI: Application to neonatal brain imaging,” Magn. Reson. Med., vol. 79, no. 3, pp. 1365-1376, 2018, L. Cordero-Grande, R. Teixeira, E. Hughes, J. Hutter, A. Price, Hajnal, “Sensitivity Encoding for Aligned Multishot Magnetic Resonance Reconstruction,” IEEE Trans. Comput. Imaging, vol. 2, no. 3, pp. 266-280, 2016 and M. W. Haskell, S. F. Cauley, and L. L. Wald, “TArgeted Motion Estimation and Reduction (TAMER): Data consistency based motion mitigation for MRI using a reduced model joint optimization,” IEEE Trans. Med. Imaging, vol. 37, no. 5, pp. 1253-1265, 2018. For the latter, the motion and image vector are jointly estimated via an inversion of the non-linear forward model. This corresponds to a large-scale non-linear optimization problem that may be computationally expensive. Previously proposed methods alternate between optimizing just the image or the motion parameters while assuming the other to be fixed (see, e.g., L. Cordero-Grande in Magn. Reson. Med.), instead of updating all optimization variables at once. Nevertheless, repeated updates of the imaging voxels lead to excessive computation that prohibits its use in clinical settings.

When the “imaging+motion” model and the underlying imaging protocol also include parallel imaging techniques that make use of the complex sensitivity profiles of multi-channel coil arrays, such as SENSE (SENSitivity Encoding) or ASSET (Array coil Spatial Sensitivity Encoding), it is referred to as “SENSE+motion” model.

In “Scout accelerated motion estimation and reduction (SAMER)”, Magn. Reson. Med., vol. 87, pp. 163-178, 2022, https://doi.org/10.1002/mrm.28971, D. Polak, D. N. Splitthoff, B. Clifford, W.-C. Lo, S. Huang, J. Conklin, L. L. Wald, K. Setsompop and S. Cauley propose a technique that utilizes a single rapid scout scan to drastically reduce the computational cost of motion estimation. The scout image contains center of k-space information that is compared against the k-space data of the actual MR acquisition for each shot to derive the subject's motion. This corresponds to registration of the k-space data with the scout image in k-space. This strategy is used to completely avoid the alternating optimization of subject motion and image volume, which is otherwise required in navigator-free, tracking-free retrospective motion correction techniques. In the SAMER-technique, a motion trajectory of the subject is first estimated, and the motion trajectory is then used in a motion-aware parallel image reconstruction, using, e.g., a “SENSE+motion” forward model, to yield the motion-mitigated image. This reduces the computational cost by several orders of magnitude, when compared to established alternating optimization methods.

In D. Polak, J. Hossbach, D. N. Splitthoff et al. “Motion guidance lines for robust data consistency-based retrospective motion correction in 2D and 3D MRI”, Magn. Reson. Med. 2023:1-14, doi: 10.1002/mrm.29534, D. Polak et al. have extended the SAMER method to include the repeated acquisition of a small number of motion guidance lines in each shot, which are used for motion estimation by being compared with the data from the scout scan. This allows for very rapid and fully separable estimation of motion parameters shot-by-shot.

However, the SAMER method using additional motion guidance lines has the disadvantage that the additional motion guidance lines require additional scan time. In multi shot sequences (e.g., MPRAGE) the repetition time TR (e.g., ˜2 sec) may be much larger than the echo train duration (˜1 sec), therefore playing additional guidance lines during the gap time of each TR does not increase the overall scan. By contrast, steady state sequences like T2*GRE constantly acquire data and hence there is no gap time which one may exploit for the guidance line acquisition. Long TEs further exacerbate the time penalty of the guidance lines.

### SUMMARY AND DESCRIPTION

It is therefore an object of the present disclosure to overcome the problems of the SAMER method, in particular the problem of prolonging the acquisition times of the imaging protocol. It is a further object to provide a method for acquiring MR image datasets for retrospective motion correction, which provides additional motion information.

The scope of the present disclosure is defined solely by the appended claims and is not affected to any degree by the statements within this summary. The present embodiments may obviate one or more of the drawbacks or limitations in the related art.

These objects are met by a method for acquiring a magnetic resonance image dataset of a field-of-view including a body part of a subject, using a gradient-echo imaging protocol, in which spatial encoding is performed using phase encoding gradients along at least one phase encoding direction, and frequency encoding gradients along a frequency encoding direction, wherein k-space is sampled during the imaging protocol by using RF excitations followed by an echo time resulting in a gradient echo, wherein during each gradient echo, an imaging k-space line oriented along the frequency encoding direction is sampled. The method includes acquiring additional k-space lines within a central region of k-space at intervals throughout the imaging protocol, the additional k-space lines being used for estimating motion parameters of the field-of-view. The gradient-echo imaging protocol has been amended by inserting additional gradient blips after at least some of the RF excitations, such that at least one additional gradient echo is generated, allowing the acquisition of at least one additional k-space line during one echo time.

The additional k-space lines are herein also referred to as “motion guidance lines” or “guidance lines.” “Additional” means that the k-space lines are acquired in addition to those that are required under the imaging protocol to acquire the MR image, i.e., they are redundant when it comes to image reconstruction.

Gradient echo (GRE) magnetic resonance imaging is a common imaging technique used for in many diagnostic settings. The disclosure exploits the dead-time between the RF excitation and the data acquisition, or between data acquisition and the next RF excitation, by inserting an additional gradient echo after the RF excitation, for example at an echo time in a range of 2 to 20 ms, in a range of 3 to 10 ms, or 4 ms. This does not increase scan time or change the contrast of the imaging data. Additional gradient encoding blips are therefore inserted in order to allow the additional k-space line and an imaging k-space line to be collected from different k-space locations.

Depending on the length of the echo time, the additional gradient echo may be acquired between the RF excitation and the acquisition of the imaging data. Alternatively, in particular in gradient echo sequences with small/very small TE, the guidance lines may also be placed after the imaging echo. In other words, in this alternative, the additional gradient echo is acquired after the acquisition of the imaging data.

The gradient echo sequence may be a steady-state sequence. It may not be a multi shot sequence (e.g., MPRAGE), in which the repetition time TR is much larger than the echo train duration.

Susceptibility weighted imaging (SWI) is a variant of GRE using long TEs. SWI is part of routine clinical brain exams but also plays an important role in emerging specialized screening protocols, such as for the detection of ARIA (amyloid related imaging abnormalities) in Alzheimer's disease patients. However, the long echo times needed to produce T2*/SWI contrast cause long acquisition times and thus increase the susceptibility to patient motion, e.g., leading to significant degradation in image quality. Therefore, the insertion of additional k-space lines has so far not been considered, because it will further increase the acquisition times. However, gradient-echo susceptibility-weighted (GRE-SWI) MR images may be acquired with a long TE to provide sufficient T2* contrast. The disclosure exploits the dead time between the RF excitation and the data acquisition, or between data acquisition and the next RF excitation, for inserting an additional gradient echo.

The imaging protocol is a susceptibility-weighted gradient-echo imaging protocol. SWI-MRI uses a type of MRI sequence that exploits the magnetic susceptibility differences of various tissues and their compounds, including de-oxygenated blood, iron, and calcium, to enable contrast in MRI. SWI is particularly useful in investigating trauma and acute neurologic occurrences such as strokes in the brain but may also be used to diagnose low-flow vascular malformations, cerebral micro-bleeds, and brain tumors. The SWI imaging protocol may have a long echo time (TE) in a range of 10 ms to 100 ms (at low field strengths of, e.g., 0.5T), in a range of 15 ms to 50 ms, in a range of 18 ms to 25 ms, or in a range of 20 ms to 30 ms (e.g., at 3 Tesla). It may be a gradient echo sequence. The repetition time (TR) may be in a range of 5 ms to 15 ms or in a range of 8 ms to 12 ms longer than the TE. The flip angle may be in a range of 5° and 25° or in a range of 10° and 20°. The imaging protocol may be flow-compensated. The main magnetic field strength may be any commercially used field strength, for example, in a range of 0.5 T and 7T. The disclosure may be applied to 3D GRE-SWI imaging protocols.

The thereby acquired susceptibility-weighted magnetic resonance image dataset, also referred to as “image dataset,” or “image,” is designed to be reconstructed using retrospective motion correction techniques, in particular the technique disclosed by D. Polak et al. in Magn. Reson. Med., vol. 87, pp. 163-178, 2022 and Magn. Reson. Med. 2023:1-14, doi: 10.1002/mrm.29534. The image dataset may be acquired for diagnostic purposes and thus may have a high spatial resolution of, for example, an in-plane resolution in a range of 0.3-3 mm or in a range of 0.4-2 mm. The voxel size may be in a range of 0.5 to 12 mm³ or in a range of 2 to 8 mm³. For a 2D image, the in-plane resolution may be in a range of 0.3-2 mm or in a range of 0.4-1.2 mm.

The imaging protocol may be a two-dimensional (2D) or three-dimensional (3D) imaging protocol. In a 2D-imaging protocol, a slice of the object may be selected during RF excitation using a slice-select gradient. During each shot, the k-space plane is sampled by using a phase encode gradient along one dimension and a frequency encoding gradient along another spatial dimension. In a 3D-imaging protocol, a larger volume of tissue is excited together. Spatial encoding is performed using phase-encoding gradients along two spatial dimensions, a partition direction and a phase-encode direction, and frequency encoding along the third dimension, referred to as readout direction. Accordingly, with each MR echo, a k-space line oriented along the readout direction is sampled, at varying partition (PAR) and phase-encode (PE) positions.

By modifying the phase-encoding gradient before each readout, it is possible to design different sampling orders (also referred to as “reordering,” “reordering scheme,” “sampling scheme,” or “reordering pattern”), which may refer to the temporal order in which the k-space lines are acquired. For example, in a linear sampling scheme, the phase-encoding gradient(s) is/are increased incrementally or step-by-step for each k-space line, resulting in a sampling of k-space line-by-line, from one end of k-space to the other. The data acquisition in 3D GRE-SWI may employ a linear reordering scheme, e.g., it iterates over all partition encoding lines (inner loop), before continuing with the next phase encoding position (outer loop).

According to an embodiment, the method includes acquiring a low-resolution scout image data set of the field-of-view. Thereby, the scout may serve as a reference when estimating the motion states from the motion guidance lines. The scout is acquired so fast that it may be assumed to represent a single motion state (i.e., the subject has not moved during the scout acquisition). The low-resolution scout image may cover the same field-of-view as the magnetic resonance image dataset. Thus, in case of a 3D image, it covers the same volume. In case the image dataset is a stack of 2D slices, the scout image also includes a stack of low-resolution 2D slices. However, it is possible that the low-resolution scout includes fewer slices than the high-resolution image, for example, only every second or third slice. Similarly, it is possible that the scout covers a larger volume for a better stability in the motion detection act. The low-resolution scout image may have a spatial resolution in a range of 2-8 mm, in a range of 3-5 mm, or 4 mm, in the phase-encoding direction(s). The acquisition of the scout may be rapid, requiring, e.g., 1-5 seconds or 1-2 seconds. It may be acquired once before or after the imaging protocol, e.g., before. Thus, the acquisition of the low-resolution scout may be the first method act, though it may also be the last or may be performed in the middle of the imaging protocol. In a further act, the imaging protocol is carried out. It is advantageous for the scout image to be contrast matched to motion guidance lines, i.e., using the same TE and TR. Thereby the motion states may be accurately estimated.

Before executing the method, an MR examination may include preparatory scans. In particular, SENSE+motion reconstructions require a coil sensitivity map that may be computed from a separate, rapid reference scan. This scan may be the first scan of the acquisition, followed by the low-resolution scout and the imaging protocol according to an embodiment.

The additional k-space lines are disposed in the central region of k-space. The central region may be equivalent to a magnetic resonance image of low resolution, such as the scout image. In particular, the central region of k-space may be equivalent to a low-resolution image having a pixel/voxel size of ≥3 mm, ≥4 mm, in a range of 3-7 mm, or in a range of 4-6 mm. It has been demonstrated to be advantageous for the guidance lines to be chosen close to the k-space center, because low-frequency k-space information may better capture the effect from patient motion than information from the k-space periphery. Moreover, in case the SAMER technique (Magn. Reson. Med., pp. 163-178, 2022) is used, the calibration samples may be chosen such that there is sufficient overlap between the scout and the calibration data for detecting motion, in particular sufficient common frequency overlap.

The method may be executed on any medical or other MRI apparatus. The field-of-view includes a body part of a subject, which may be human or animal, in particular a patient to be examined. The image dataset is in particular acquired from a part of the body that is subject to undesired motion, for example the head or neck, a limb such as a leg, arm, knee, hand, or a part subjected to breathing motion such as the thorax or abdomen.

The additional k-space lines are used for estimating motion parameters of the field-of-view. The motion parameters are in particular a so-called motion trajectory, i.e., the motion parameters describe the position of the body part of the subject within the field-of-view at various time points throughout the imaging protocol, wherein the time points are separated by intervals. The method allows for acquiring a motion guidance line (additional k-space line) every TR, which may be every 10-100 ms or every 20-50 ms, i.e., repetition times (TR) for a SWI imaging protocol at 3T. In certain embodiments, a motion state is not estimated from a single motion guidance line, but from a set in a range of 2-16 or in a range of 4-8 motion guidance lines sampling different positions within the central region of k-space. However, even when using a set of motion guidance lines of each motion state, the temporal resolution which is attainable by using the deadtime before or after the imaging echo, is more than enough for the purpose of motion trajectory estimation. Therefore, in some embodiments, additional k-space lines are not acquired during each repetition time, but only in a portion thereof, for example, in a range of 5-60% or in a range of 10-40% of the repetition times. This reduces the patient exposure to imaging gradients, thereby reducing the gradient switching noises and associated energy consumption for the imaging protocol. In other embodiments, additional k-space lines are acquired at each repetition time and are used for deducing further information, for example to estimate the BO-field inhomogeneities, to acquire an or another low-resolution image data set of the field-of-view, or to increase the time resolution of the estimated motion parameters.

According to an embodiment, the additional k-space lines are acquired from a set in a range of 2 to 16 or in a range of 4 to 8 k-space positions within a central region of k-space, wherein each set in a range of 2 to 16 or in a range of 4 to 8 additional k-space lines is used to estimate one motion state of the field-of-view, e.g., a rigid-motion state. A rigid motion state may be described by a number of parameters, in particular three translation parameters and three rotation parameters. Each set of additional k-space lines may include fewer k-space lines than would be required to reconstruct a scout image. The set of additional k-space lines may be acquired repeatedly during the imaging protocol, e.g., at least approximately, at regular intervals. “At least approximately,” when used herein, may mean within +15%, within +10%, or within +5%. Each set of additional k-space lines is intended to provide information on the position of the subject at the point in time at which the set was acquired, also referred to as motion state. Therefore, the interval at which the sets of additional k-space lines are acquired may be in a range of 0.3 s and 3.0 s, in a range of 0.5 s and 2.0 s, or in a range of 1.0 s and 2.0 s. By acquiring sets of guidance lines in more TRs, a motion state may even be estimated every 0.2-0.8 seconds. The sets of additional k-space lines may be at the same position in k-space at each acquisition, but this is not mandatory.

The density of the additional k-space lines may vary during the acquisition, e.g., in order to be able to determine motion parameters with a higher temporal resolution for important k-space parts more (e.g. k-space center), or in case the already detected motion parameters during the acquisition indicate a higher temporal resolution is required.

Each set of additional k-space lines (motion guidance lines) may be acquired from consecutive RF excitations. Thereby, it is assured that each set represents one motion state of the subject, by minimizing the time delay between the acquisition of the additional k-space lines which are used to estimate one motion state.

According to an embodiment, the susceptibility-weighted gradient-echo imaging protocol is a 3D imaging protocol using a linear sampling order, wherein one set of 2-16 or 4-8 additional k-space lines is acquired during each iteration of an inner phase-encoding loop of the imaging protocol. The inner phase-encoding loop may be the partition encoding, in case of imaging protocols which iterate over all partition encoding lines in an inner loop, before continuing with the next phase encoding position in an outer loop. In order to achieve a temporal resolution of 1-2 seconds, e.g., one set of, for example, four motion guidance lines may be collected during every run of the inner loop.

According to an embodiment, at least one additional k-space line is acquired during each repetition time, i.e., after each RF excitation, in particular if the echo time of the imaging protocol is long enough to accommodate at least one k-space line or two or three k-space lines, as for example in SWI imaging. Thus, embodiments in which (a) at least one additional k-space line is acquired after each RF excitation, and/or (b) two or three additional k-space lines are acquired after at least some RF excitations, are conceivable. These additional k-space lines may be used for a higher temporal resolution of the motion estimation. In certain examples, the additional k-space lines may be used to provide additional input, for example, to estimate BO field variations. This is because every guidance line echo acquired per excitation is acquired at a slightly different echo time, but at the same k-space location, and thus the change in signal across the echoes reflects the BO inhomogeneity. Advantageously, this data is acquired from multiple k-space locations to get enough spatial encoding. According to an embodiment, of two separate low-resolution images are acquired, using the additional k-space lines, at different TEs for generating a BO field map.

Furthermore, the acquisition of the motion guidance lines may be interleaved with low-resolution images or navigators for additional pose estimation, or for estimating field homogeneities associated with the motion. In other words, only some of the additional k-space lines acquired per RF excitation are used as motion guidance lines to estimate motion states. Other additional k-space lines are used to acquire a low-resolution image or navigator. Furthermore, they may be used to estimate homogeneities in the BO-field that are also caused by motion, and for which may be compensated.

According to an embodiment, the phase of at least some of the additional k-space lines is used to estimate the BO-field inhomogeneities caused by motion. This may, for example, be done using the approach described by Y. Brackenier in “Data-driven motion-corrected brain MRI incorporating pose-dependent BO-fields,” Magn. Reson. Med., vol. 88, no. 2, pp. 817-831, 2022. Therein, the data-driven retrospective motion correction includes modelling of pose-dependent changes in the BO-field in the head. The use of a physics-based BO-field model constrains the number of unknowns to be found, enabling motion correction based on data-consistency. Thus, the BO-fields may be estimated based on data-consistency by amending the SENSE+motion model by including an additional phase term ein into the forward model which may vary per motion state. Alternatively, the discrete Fourier operator may be replaced with a field-dependent Fourier operator.

According to an embodiment, the imaging protocol includes flow compensation gradients inserted between the acquisitions of the imaging k-space lines and the additional k-space lines, in order to reduce flow artefacts in the magnetic resonance image data set. Flow compensation gradients are known in the art. However, flow suppression is only required for the imaging echo, because the motion guidance lines are used for motion trajectory estimation only. Therefore, the flow compensation gradients may be inserted as close as possible before the acquisition of the imaging k-space lines, i.e., the data used for image reconstruction. This position is beneficial, because it reduces the required peak gradient strength and potential nerve stimulation. The flow compensation gradients may be configured to achieve full flow suppression in the imaging echo (e.g., imaging k-space lines), taking into account all guidance lines and imaging gradients.

According to a further aspect, a method for generating a motion-corrected magnetic resonance image dataset of an object is provided. The method includes receiving k-space data acquired using the acquisition method according to an embodiment, and receiving a low-resolution scout image dataset and sets of additional k-space lines acquired using the acquisition method according to an embodiment.

The motion correction algorithm includes, in a first act, estimating motion parameters for each set of additional k-space lines by minimizing the data consistency error between the additional k-space lines and a forward model using the low-resolution scout scan as an estimate for the image dataset, wherein the forward model is described by an encoding matrix including the motion parameters to be estimated, Fourier encoding, and optionally subsampling and/or coil sensitivities of a multi-channel coil array.

In a second act, the motion correction algorithm includes estimating the motion-corrected image dataset by minimizing the data consistency error between the k-space data acquired in the imaging protocol and a forward model described by an encoding matrix, wherein the encoding matrix includes the motion parameters estimated in the first act for each set of additional k-space lines, Fourier encoding, and optionally subsampling and/or coil sensitivities of a multi-channel coil array.

The motion parameters may be rigid-body motion parameters, including, e.g., three translational and three rotational parameters, or non-rigid motion parameters.

Thus, the minimization problem is carried out in two acts. In a first act, motion parameters are estimated for each set of guidance lines by minimizing the data consistency error of the forward model, which may amount to a comparison with the low-resolution scout image. In a second act, the motion-corrected image is estimated using the motion parameters estimated in the first act. If motion dependent BO fields were estimated, as explained above, this information may also be included into the image reconstruction/image estimation. Thereby, alternating repeated updates of the otherwise coupled optimization variables x (image vector) and θ (motion parameters) is avoided. Rather, the rapid scout image dataset is used as an image estimate x. This leads to a highly efficient optimisation problem that is fully separable across the echo trains and does not require repeated updates of x, which may include millions of imaging voxels. The minimisation problem may be derived from a SENSE parallel imaging forward model, as described in K. P. Pruessmann, M. Weiger, M. B. Scheidegger, and P. Boesiger, “SENSE: sensitivity encoding for fast MRI,” Magn. Reson. Med., vol. 42, no. 5, pp. 952-962, 1999, with rigid body motion parameters included (“SENSE+motion” model). The overall method is described in Magn. Reson. Med. 2023:1-14, doi: 10.1002/mrm.29534, which is incorporated herein by reference.

In a further aspect, a magnetic resonance imaging apparatus is provided, which includes a radio frequency controller configured to drive an RF-coil, e.g., including a multi-channel coil-array, a gradient controller configured to control gradient coils, and a control unit configured to control the radio frequency controller and the gradient controller to execute the imaging protocol. The MRI-apparatus may be a commercially available MRI-apparatus which has been programmed to perform the method.

According to a further aspect, a computer configured to generate a motion-corrected magnetic resonance image dataset is provided. The computer may be any computer including a sufficiently powerful processing unit, which may be a CPU or GPU, or several such processing units. Accordingly, the computer may be a PC, a server, a console of an MRI apparatus, but it also may be a computer that is remote from the MRI apparatus, it may be connected with it through the internet. Accordingly, the computer may also be a cloud computer, a remote server, etc. The computer may also be a mobile device, such as a laptop, tablet computer or mobile phone.

According to a further aspect, a computer program is provided which includes program code, which causes a magnetic resonance imaging apparatus, such as the apparatus described herein, to execute the method, in particular the method for acquiring an MR image dataset. However, the program code may also encode the described method for generating a motion-corrected magnetic resonance image dataset, and the program code may run on a computer as described herein.

According to a further aspect, the disclosure is directed to a non-transitory computer-readable medium containing a computer program as described herein. The computer-readable medium may be any digital storage medium, such as a hard disc, a cloud, an optical medium such as a CD or DVD, a memory card such as a compact flash, memory stick, a USB-stick, multimedia stick, secure digital memory card (SD), etc.

All features disclosed with regard to the acquisition method may be combined with all features of method for generating a motion-corrected MRI dataset and vice versa. Also, all features of the disclosed methods may be embodied in the MRI apparatus, computer program and computer-readable storage medium according to other aspects of the disclosure and vice versa.

### BRIEF DESCRIPTION OF THE DRAWINGS

The accompanying drawings illustrate various example embodiments of various aspects of the disclosure.

FIG. 1 is a schematic representation of an MRI apparatus according to an embodiment.

FIG. 2 is a schematic representation of an example of a three-dimensional k-space.

FIG. 3 is a sequence diagram of an example of a SWI sequence with motion guidance lines.

FIG. 4 is an example image of a k-space illustrating the temporal acquisition order of the imaging k-space lines.

FIG. 5 is an illustration of an example of an alternating optimization algorithm.

FIG. 6 is an illustration of a SAMER optimization.

Similar elements are designated with the same reference signs in the drawings.

### DETAILED DESCRIPTION

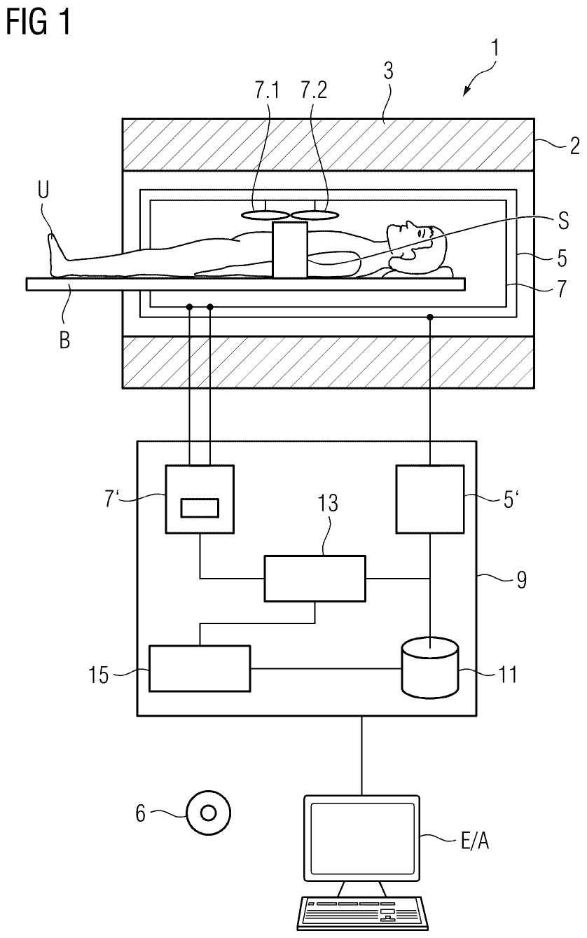

FIG. 1 schematically shows an example of a magnetic resonance (MR) apparatus 1. The MR apparatus 1 has an MR data acquisition scanner 2 with a main magnet 3 that generates the constant magnetic field Bo, a gradient coil arrangement 5 that generates the gradient fields, one or several radiofrequency (RF) antennas 7 for radiating and receiving RF signals, and a control computer 9 configured to perform the method. The radio-frequency antennas 7 may include a multi-channel coil array including at least two coils, for example the schematically shown coils 7.1 and 7.2, which may be configured to transmit and/or receive RF signals (MR signals).

In order to acquire MR data from an examination subject U, for example, a patient or a phantom, the subject U is introduced on a bed B into the sensitive volume or bore of the scanner 2. A 3D MR image may be acquired using a method according to an embodiment from a 3D volume S. The control computer 9 controls the MR apparatus 1, and the gradient coil arrangement 5 with a gradient controller 5′ and the RF antenna 7 with a RF transmit/receive controller 7′. The RF antenna 7 has multiple channels corresponding to the multiple coils 7.1, 7.2 of the coil arrays, in which signals may be transmitted or received. The control computer 9 also has an imaging protocol processor 15 that determines the imaging protocol, including the acquisition of imaging and additional k-space lines. A control unit 13 of the control computer 9 is configured to execute all the controls and computation operations required for acquisitions. Intermediate results and final results required for this purpose or determined in the process may be stored in a memory 11 of the control computer 9. A user may enter control commands and/or view displayed results, for example image data, an input/output interface E/A. A non-transitory data storage medium 6 may be loaded into the control computer 9 and may be encoded with programming instructions (program code) that cause the control computer 9, and the various functional units thereof described above, to implement any or all embodiments of the method.

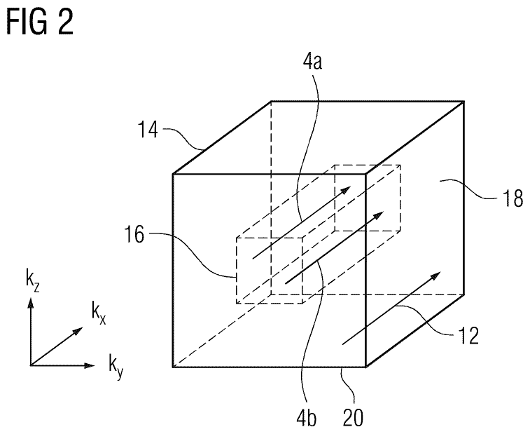

FIG. 2 illustrates a three-dimensional k-space 14 having dimension kx in frequency encoding (readout) direction, and ky and kz in the phase encode plane 20. The k-space volume 14 includes a central region 16 and a periphery 18. An imaging k-space line 12 acquired during one echo is illustrated in the periphery. On the other hand, additional k-space lines 4a, 4b as well as the k-space lines acquired for the low-resolution scout are located in the central region 16. In the phase encode plane 20, the central region 16 may cover about 1/12 to 1/16 in each direction, i.e., less than 1/100 of the total square phase encode plane 20.

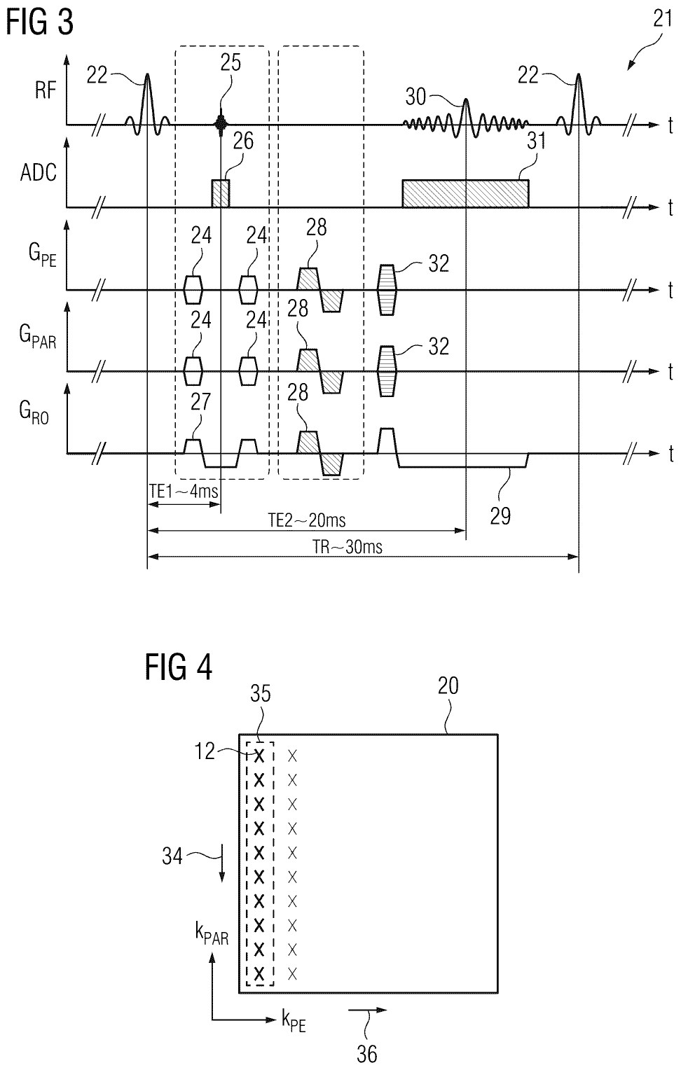

FIG. 3 illustrates a T2*-weighted imaging protocol 21 including the acquisition of motion guidance lines 4 according to an embodiment.

The diagram depicts the RF-pulses and signals on the top line, the ADC (analogue-to-digital converter) on the line below. This indicates when data is acquired. Below that are two lines for the two-phase encoding gradients, wherein GPE indicates the phase encoding direction and GPAR the partition direction. The frequency encoding (readout) gradients are shown below. The diagram 21 illustrates one repetition time TR, which in this case is 30 ms long. At the beginning of each TR, an RF excitation pulse 22 is transmitted. This is followed by gradient blips 24, which are used to direct the signal to one of the central positions in the phase encode plane 20, in which the motion guidance lines are acquired. The acquisition of an additional k-space line 4 in the shape of a guidance line echo 25 is indicated by the ADC at 26, and by the readout gradient 27. In the implemented example, the echo time TE1 of the guidance line echo, 25, was 4 ms. Flow compensation gradients are played just before the acquisition of the imaging echo 30. Note that flow compensation gradients along the frequency encoding (readout) direction may also be played out but are not shown in this diagram. Thus, after the flow compensation gradients 28, phase encode gradient blips 32 are played out in order to adjust the position of the imaging k-space lines 12 in the phase-encode plane 20, before the ADC 31 is switched on for the acquisition of the imaging echo 30 during readout gradient 29 at an echo time TE2 of 20 ms in this example. At 10 ms after the last echo, the TR is repeated with RF pulse 22.

FIG. 4 illustrates the linear sequence ordering in GRE-SWI, wherein the acquisition of the imaging k-space lines 12 iterates through all partition encoding acts in an inner loop, indicated by arrow 34, before moving to the next phase encoding position, i.e., the next line along the outer loop direction 36. In an example, four additional k-space lines (guidance line echoes) are collected during each run of the inner loop, which is illustrated by the box 35. This enables motion trajectory estimation once per second.

The sequence illustrated in FIG. 3 has been implemented and has been shown to yield improved image sharpness and vessel depiction in situations with step motion and breathing motion. Further, this SWI sequence with guidance lines and flow compensation according to an embodiment has achieved excellent image quality and flow artefact suppression, in addition to motion compensation.

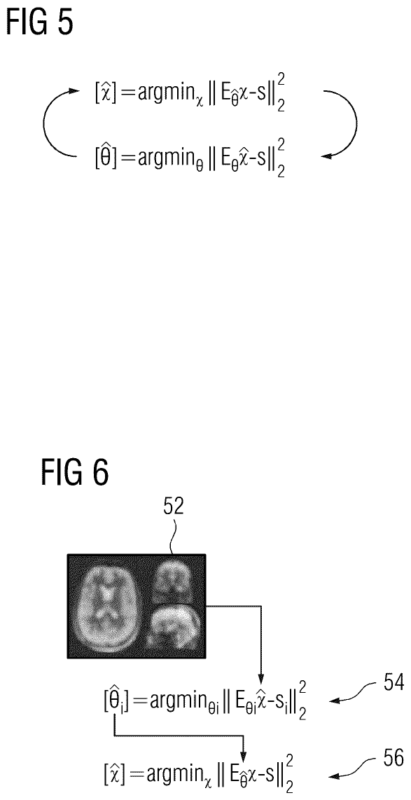

A retrospective motion correction technique, which may be used on the MR signals acquired with the acquisition method, e.g., to estimate the motion states, is described with reference to FIGS. 5 and 6. The mathematical model used is an extension of SENSE parallel imaging, as described in the above-cited paper by K. P. Pruessmann et al., with rigid-body motion parameters included into the forward model. The forward model or encoding operator E_{θ} for a given patient motion vector θ (e.g., including motion parameters over time) relates the motion-free image x to the acquired multi-channel k-space data s. At each time point i that is considered, e.g., the acquisition time of the sets of guidance lines, the subject's position is described by a new set of six rigid-body motion parameters θᵢ that describe the 3D position of the object. Accordingly, the multi-channel k-space data sᵢ acquired at time point i may be related to the 3D image volume x through image rotations Rᵢ, image translations Tᵢ, coil sensitivity maps C, Fourier operator F and under-sampling mask Mᵢ by the following formula 1:

$${s}_{i} = {E}_{{θ}_{i}} x = {M}_{i} F C {T}_{{θ}_{i}} {R}_{{θ}_{i}} x \qquad [ 1 ]$$

In prior art methods, as illustrated in FIG. 5, both the motion corrected image vector x and the motion vector (trajectory) θ are estimated by performing an alternating, repeated optimization between the image vector (formula 2) and the motion vector (formula 3):

$$[ \hat{x} ] = arg {min}_{x} {‖ {E}_{\hat{θ}} x - s ‖}_{2} \qquad [ 2 ]$$

$$[ \hat{θ} ] = arg {min}_{θ} {‖ {E}_{θ} \hat{x} - s ‖}_{2} \qquad [ 3 ]$$

This may lead to convergence issues as updates of x and 0 will be computed on inaccurate information. Moreover, the reconstruction is computationally demanding as repeated updates of x (millions of imaging voxels) are needed.

Using one or several ultra-fast low-resolution scout scans 52, the motion trajectory may be directly estimated, as illustrated in FIG. 6, thus avoiding the time-consuming alternate optimization. The scout image 52, designated by \tilde{x}, approximates the motion-free image volume \hat{x} and each motion state may be determined independently by minimizing the data consistency error of the forward model 54:

$$[ {\hat{θ}}_{i} ] = arg {min}_{{θ}_{i}} {‖ {E}_{{θ}_{i}} \tilde{x} - {s}_{i} ‖}_{2} \qquad [ 4 ]$$

In the method, the guidance lines are used instead of or in addition to the k-space data s in this first act of the optimization. In certain examples, only the guidance lines are used.

For the final image reconstruction, the motion states/parameters from each set of guidance lines are combined and the motion-mitigated image is obtained from solving a standard least-squares problem 56:

$$[ \hat{x} ] = arg {min}_{x} {‖ {E}_{\hat{θ}} x - s ‖}_{2} \qquad [ 5 ]$$

This strategy completely avoids the difficult non-linear and non-convex joint optimization that contains millions of unknowns, as it only considers six rigid body parameters per motion optimization, and it does not require computationally costly full or partial updates to the image.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for acquiring a magnetic resonance image dataset of a field-of-view comprising a body part of a subject, the method comprising:
using a gradient-echo imaging protocol, in which spatial encoding is performed using phase encoding gradients along at least one phase encoding direction and frequency encoding gradients along a frequency encoding direction;
sampling k-space during the gradient-echo imaging protocol by using radio frequency (RF) excitations followed by an echo time resulting in a gradient echo, wherein, during each gradient echo, an imaging k-space line oriented along the frequency encoding direction is sampled; and
acquiring additional k-space lines within a central region of k-space at intervals throughout the gradient-echo imaging protocol,
wherein motion parameters of the field-of-view are estimated using the additional k-space lines, and
wherein the gradient-echo imaging protocol is amended by inserting additional gradient blips after at least some of the RF excitations such that at least one additional gradient echo is generated, allowing for an acquisition of at least one additional k-space line during one echo time.

2. The method of claim 1, wherein the gradient-echo imaging protocol is amended by inserting the additional gradient blips between at least some of the RF excitations and the corresponding gradient echoes.

3. The method of claim 1, wherein the additional k-space lines are acquired from a set of 2 to 16 k-space positions within the central region of k-space, and
wherein each set of 2 to 16 additional k-space lines is used to estimate a motion state of the field-of-view.

4. The method of claim 3, wherein the motion state is a rigid-motion state.

5. The method of claim 1, wherein the additional k-space lines are acquired from a set of 4 to 8 k-space positions within the central region of k-space, and
wherein each set of 4 to 8 additional k-space lines is used to estimate a motion state of the field-of-view.

6. The method of claim 1, wherein the gradient-echo imaging protocol is a steady-state gradient-echo imaging protocol, a susceptibility-weighted gradient-echo imaging protocol, or a combination thereof.

7. The method of claim 6, wherein the susceptibility-weighted gradient-echo imaging protocol is a three-dimensional imaging protocol using a linear sampling order, and
wherein one set of 2 to 16 additional k-space lines is acquired during each iteration of an inner phase-encoding loop of the susceptibility-weighted gradient-echo imaging protocol.

8. The method of claim 6, wherein the echo time of the susceptibility-weighted gradient-echo imaging protocol is in a range of 10 ms to 100 ms.

9. The method of claim 1, wherein at least one additional k-space line is acquired during each echo time.

10. The method of claim 1, wherein the additional k-space lines are used to estimate pose-dependent BO-field variations.

11. The method of claim 1, wherein at least one additional k-space line of the additional k-space lines is used to acquire a low-resolution image dataset of the field-of-view.

12. The method of claim 1, wherein the gradient-echo imaging protocol comprises flow compensation gradients inserted between the acquisitions of the additional k-space lines and the imaging k-space lines, in order to reduce flow artefacts in the magnetic resonance image dataset.

13. A method for generating a motion-corrected magnetic resonance image dataset of an object, the method comprising:
receiving k-space data;
receiving a low-resolution scout image dataset and sets of additional k-space lines;
estimating motion parameters for each set of additional k-space lines by minimizing a data consistency error between the additional k-space lines and a forward model using the low-resolution scout scan as an estimate for the image dataset, wherein the forward model is described by an encoding matrix comprising the motion parameters to be estimated, Fourier encoding, and optionally subsampling and/or coil sensitivities of a multi-channel coil array; and
estimating the motion-corrected image dataset by minimizing the data consistency error between the k-space data acquired in a gradient-echo imaging protocol and the forward model described by the encoding matrix, wherein the encoding matrix comprises the motion parameters estimated for each set of the additional k-space lines, the Fourier encoding, and optionally the subsampling and/or the coil sensitivities of the multi-channel coil array.

14. The method of claim 13, wherein the k-space data is sampled during the gradient-echo imaging protocol by using radio frequency (RF) excitations followed by an echo time resulting in a gradient echo, wherein, during each gradient echo, an imaging k-space line oriented along the frequency encoding direction is sampled.

15. The method of claim 14, wherein the sets of additional k-space lines are acquired within a central region of k-space at intervals throughout the gradient-echo imaging protocol.

16. A magnetic resonance imaging apparatus comprising:
a radio frequency controller configured to drive an RF-coil comprising a multi-channel coil array;
a gradient controller configured to control gradient coils; and
a control unit configured to control the radio frequency controller,
wherein the gradient controller is further configured to:
use a gradient-echo imaging protocol, in which spatial encoding is performed using phase encoding gradients along at least one phase encoding direction and frequency encoding gradients along a frequency encoding direction;
sample k-space during the gradient-echo imaging protocol by using radio frequency (RF) excitations followed by an echo time resulting in a gradient echo, wherein, during each gradient echo, an imaging k-space line oriented along the frequency encoding direction is sampled; and
acquire additional k-space lines within a central region of k-space at intervals throughout the gradient-echo imaging protocol,
wherein motion parameters of a field-of-view are estimated using the additional k-space lines, and
wherein the gradient-echo imaging protocol is amended by inserting additional gradient blips after at least some of the RF excitations such that at least one additional gradient echo is generated, allowing for an acquisition of at least one additional k-space line during one echo time.
